(19) United States
(12) Patent Application Publication
(10) Pub. No.: US 2021/0322766 A1
(43) Pub. Date: Oct. 21, 2021

(54) SYSTEM AND METHOD FOR DEACTIVATING TOXINS IN SKIN

(71) Applicant: Jeffry Skiba, Chandler, AZ (US)

(72) Inventor: Jeffry Skiba, Chandler, AZ (US)

(21) Appl. No.: 17/235,713

(22) Filed: Apr. 20, 2021

Related U.S. Application Data
(60) Provisional application No. 63/012,262, filed on Apr. 20, 2020.

(51) Int. Cl. A61N 1/18; A61N 1/04; A61N 7/00

(52) U.S. Cl. CPC A61N 1/18; A61N 1/0476; A61N 2001/083; A61N 7/00; A61N 1/0484

(57) ABSTRACT

A cartridge for applying an electromagnetic field (EMF) to at least one toxin in a wound in the skin of a person or animal is provided. The cartridge includes a power supply, a controller coupled to the power supply and providing a voltage between about 0.2V and 3V to an array of electrodes configured to transmit the EMF to the skin about the wound site. A cartridge for applying an ultrasonic field to at least one toxin in a wound in the skin of a person or animal is also provided. The ultrasonic cartridge may include a power supply, a controller coupled to the power supply and providing a voltage to at least one sonotrodes configured to transmit the ultrasonic field to the skin about the wound. The cartridge may also include a sensor for measuring the resistance or impedance in the tissue to be treated and the controller can adjust the ultrasonic field based on the impedance or resistance.

### CROSS REFERENCE TO RELATED APPLICATION

This application claims benefit from currently pending U.S. Provisional Application No. 63/012,262 titled “Biologic Protein Denaturing Screen” and having a filing date of Apr. 20, 2020, all of which is incorporated by reference herein.

### FIELD OF THE INVENTION

The present specification relates generally to applying an electric field to an area of injury including one or more foreign toxins in the skin of a person or animal to deactivate the toxin.

### BACKGROUND OF THE INVENTION

Current tissue ablation techniques rely on a high-frequency, hyper-thermia inducing electric current to the tissue of a patient (e.g., human, animal, etc.) as a means to remove unwanted tissue or lesions, staunch bleeding, or cut tissue. There has been increased interest and activity is the area of hyper-thermal ablation as a tool to treat cancer by heat-induced killing and/or removal of tumor tissue. U.S. Pat. No. 8,915,911 teaches electric field delivery and non-thermal or selective ablation of target tissue regions, including selective ablation of cancerous cells and solid tumors. An electric field is applied to a tissue, including positioning an electrode within a target tissue region comprising cancerous cells, and applying an alternating electrical current to the target tissue to non-thermally ablate cancerous cells of the target tissue region around the electrodes. Numerous other methods and devices are taught using hyper-thermal or heat-induced microbe, toxin, or cancer tissue destruction. However, a significant limitation of RF induced, hyper-thermal ablation is the difficulty of localizing the heat-induced damage to targeted unwanted substances or tissue while limiting histological damage and destruction to surrounding healthy, non-target tissue.

Many toxic venoms found in nature are composed of proteins developed as part of a defense or predation mechanism. Many of these proteins have an adverse effect on animals and humans ranging from mild inflammation, to death and are commonly referred to as poisonous. These proteins are all commonly delivered by direct injection, stingers or fangs.

Venoms kill through the action of at least four major classes of toxin, such as necrotoxins and cytotoxins which kill healthy cells. Neurotoxins affect the nervous systems and myotoxins will damage the muscles. Venom can be delivered in a bite, sting, or similar action used to puncture a person's or animal's skin. While venomous animals cause tens of thousands of human deaths per year, when properly prepared the toxins in many venoms have potential to treat a wide range of diseases.

Proteins are polar molecules that are affected greatly by exposure to electrical fields and other physical energy forms such as ultrasound and light. Proteins are chemicals made up of chains of amino acids forming long chain carbons, oxygen, hydrogen and nitrogen. Proteins may be thought of as having a long chain backbone with different R groups extending from the backbone. The sequence of amino acids that make up a protein (the primary structure) folds into secondary and tertiary structures in three dimensions. Secondary structure refers to local folded structures that form within a polypeptide due to interactions between atoms of the carbon chain backbone. Secondary structures are held in shape by hydrogen bonds, which form between the carbonyl O of one amino acid and the amino H of another. Tertiary structure is largely due to interactions between the R groups of the amino acids making up the protein. R group interactions that contribute to tertiary structure include hydrogen bonding, ionic bonding, dipole-dipole interactions, and London dispersion forces—basically, a variety of interaction not involving covalent bonds. Especially complex proteins may be composed of multiple polypeptide chains that come together to give a protein a quaternary structure. In general, the same types of interactions that contribute to tertiary structure (mostly weak interactions, such as hydrogen bonding and London dispersion forces) also hold the subunits together to give quaternary structure.

The internal structures of proteins can vary widely but do contain many structures that can be manipulated with energy. A device that can alter the shape of the protein and performing this at a distance can be useful in altering the protein shape and therefore the venom that it forms a part of.

Therapeutic application of electrical fields to human tissue has been traditionally used to stimulate muscle and nerve fibers. It has been used to reduce pain via skin electrodes powered by batteries and controlling the frequency, amplitude and duration. Muscles and nerves are very large structures when compare to proteins. The electric field required to alter a venom is much less than traditional electric muscle stimulators. In addition, the shape of the electric field can be altered to optimize the field effects on proteins. The polar sections of the protein molecule chain are attracted to either the positive or negative pole when in an electric field. By altering the physical shape of the protein molecule, the poisonous effects of that venom can be altered as well.

The applied energy to the molecule can be delivered by electric field, ultrasound, light or heat. In the case of human envenomation, only electric fields and ultrasound are applicable for deep envenomation as light and heat will either damage the healthy skin or not reach deep enough to affect the venom deep in the tissue.

The application of an electric field can be accomplished using standard skin electrodes; however, the limitations of the art are in the speed of application and the device itself may be too large for the intended use. Therefore, there is a need for a device that can provide an electric field to a bite sight where venom or neurotoxin has been injected into the body wherein the device can quickly denature the protein within the bite sight.

### BRIEF SUMMARY OF THE INVENTION

In one embodiment, a cartridge for applying an electromagnetic field (EMF) to at least one toxin in a wound in the skin of a person or animal is provided. The cartridge includes a power supply, a controller coupled to the power supply and providing a voltage between about 0.2V and 3V to an array of electrodes configured to transmit the EMF to the skin about the wound site. The power supply can be a battery, a solar cell, a galvanic reaction, or the like. In a particular embodiment, the power supply is a half-cell reaction between a zinc thread and oxygen found within the skin. The electrode can be an array of electrodes formed from a woven matrix of conductive threads spaced apart a distance to create the EMF.

A cartridge for applying an ultrasonic field to at least one toxin in a wound in the skin of a person or animal is also provided. The ultrasonic cartridge may include a power supply, a controller coupled to the power supply and providing a voltage to at least one sonotrodes configured to transmit the ultrasonic field to the skin about the wound. 10. The cartridge may also include a sensor for measuring the resistance or impedance in the tissue to be treated and the controller can adjusts the ultrasonic field based on the impedance or resistance.

When the EMF is applied to the skin of a person or animal having received a venomous wound, the applied EMF operates to denature the protein found in the toxin or venom and renders it less harmful to the person or animal. A method is therefor provided in which a toxin in a wound is deactivated by providing an electromagnetic field (EMF) to the wound with a voltage between about 0.2V and 3V through an array of electrodes configured to transmit the EMF to the skin about the wound. The EMF serves to denature the protein in the toxin to render it at least partially inert to the person or animal who has sustained the wound. The voltage or ultrasonic treatment may be calibrated to effectively denature the toxin but not damage the healthy cells of the person or animal being treated.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for” and will also recite the word “function” (i.e., will state “means for performing the function of . . . , without also reciting in such phrases any structure, material or act in support of the function. Thus, even when the claims recite a “means for performing the function of molding a . . . , step for performing the function of molding a . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 shows an isometric view of an EMF cartridge and housing in accordance to one, or more embodiments;

FIG. 2 is a diagram of the EMF cartridge, controller and power source in accordance to one, or more embodiments;

FIG. 3 is a type of flexible array of small electrodes in accordance to one, or more embodiments;

FIG. 4 is an array of pin type electrodes that create electric fields between the tips of the electrodes in accordance to one, or more embodiments;

FIG. 5 is a printed electric field array using printed, dissimilar metals to create a galvanic reaction resulting in electric fields between each cell pair in accordance to one, or more embodiments;

FIG. 6 is example of a woven fiber print with different materials associated with the fibers in accordance to one, or more embodiments;

FIG. 7 is another example of a woven different weave fiber print with different materials associated with the fibers accordance to one, or more embodiments;

FIG. 8 is an example of an open weave pattern for keeping metal fibers physically separated in accordance to one, or more embodiments;

FIG. 9 is an example of a possible weave pattern for keeping metal fibers physically separated in accordance to one, or more embodiments;

FIG. 10 is another example of a possible weave pattern for keeping metal fibers physically separated in accordance to one, or more embodiments;

FIG. 11 sonitrons ultrasound in accordance to one, or more embodiments;

FIG. 12 focused ultrasound in accordance to one, or more embodiments; and

FIG. 13 focused ultrasound on a target protein using an array to focus the ultrasound at the target a in accordance to one, or more embodiments.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

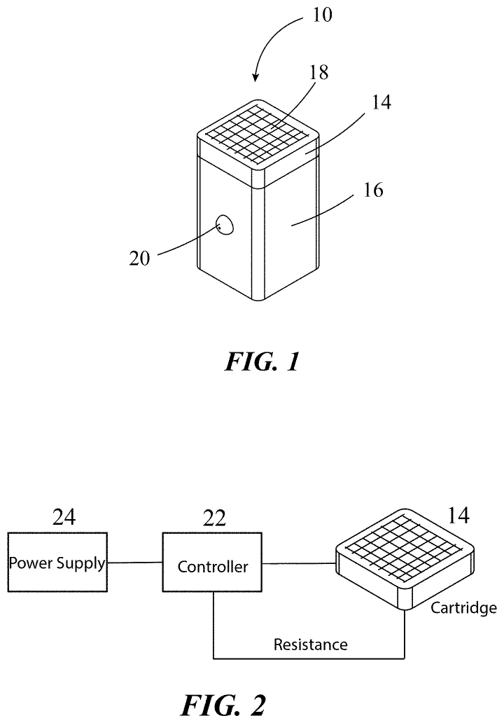

Referring initially to FIG. 1-2, in one embodiment, an electromagnetic field (EMF) is provided by an EMF cartridge 14. The EMF cartridge 14 includes a housing 16 encompassing a power supply 24 coupled through a controller 22 to at least one electrode 18. The power supply 24 may be a battery, capacitor, solar cell, galvanic reaction or other source of stored or on-demand electrical energy. The power supply 24 may be a rechargeable lithium-ion battery, a disposable battery, inductance charging or the like. In a particular embodiment, the power supply 34 is a replaceable AA or AAA alkaline battery. The EMF applied may be a direct or alternating current.

The power supply 24 is coupled to a controller 22. The controller 22 determines the frequency, amplitude, waveform shape, and duration of electricity that is supplied from the power supply 24 to the at least one electrode 18. The controller 22 is operated by a user through an interface 20. In a simple embodiment, the interface 20 includes a switch to turn the cartridge on which can be a button, switch, digital interface, or the like that allows electricity to flow from the power supply 24 to the electrode 18 when the interface such as the button is depressed. The electricity supplied from the power supply 24 to the at least one electrode 18 is between 0.2 V and 3V. This voltage can be either alternating current or direct current and the application time of the voltage can be frequency modulated.

The at least one electrode 18 is comprised of multiple cathodes and anodes separated by a maximum of about 1 mm. In a particular embodiment, the total area of the at least one electrode 18 is about one square inch. By shrinking the electric field electrodes 18 to a small matrix, the applied field can be shaped in applied depth and field strength. An array of microelectrodes 18 where the electrode pattern is predetermined against living tissue can project smaller, more numerous electric fields into the tissue contacted. In another embodiment, the use of tissue impedance measuring can be used to modulate the delivery of the electrical field or ultrasound. This feature would allow the device to measure the affected area for impedance that would be affected by the presence of venom or any other foreign substance and compare that to the normal tissue impedance. Treating the area with the device 10 until the impedance is matched would allow the device to be interactive with the tissue and control the device as it delivers the energy to the area.

The controller 22 may include filtration media. The filtration media may be actively powered with low level voltage either as point charges, as a single sided battery cell that works only when in combination with air and humidity within the active flow, or a double or multi celled battery that can produce a set or variable voltage. This voltage can be either alternating current or direct current and the application time of the voltage can be frequency modulated. Proteins are polar in nature and can be collapsed with the application of the correct level of electricity or electrical charge. This invention could be used as an addition to air filters found in hospitals or home where an airborne microbe or virus might be found. It could also be used within face mask media used to provide protection from airborne contamination.

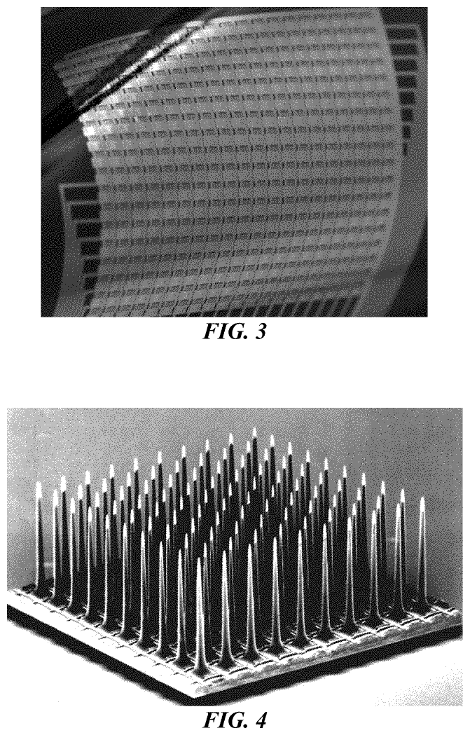

Referring to FIG. 3 shows an example flexible array of electrodes. In certain embodiments, the top of the EMF cartridge 14 can be flat surface or have a curvature to it wherein the flexible array of electrodes which can take on any shape of the EMF cartridge 14 as shown in FIG. 1.

FIG. 4 shows an array of pin type electrodes that create EMF between the tips of the electrodes wherein the pin type electrodes can be on the top of the EMF cartridge wherein when the pin type of electrodes can be pushed into the user's would, bite or sting and create EMF within the tissue.

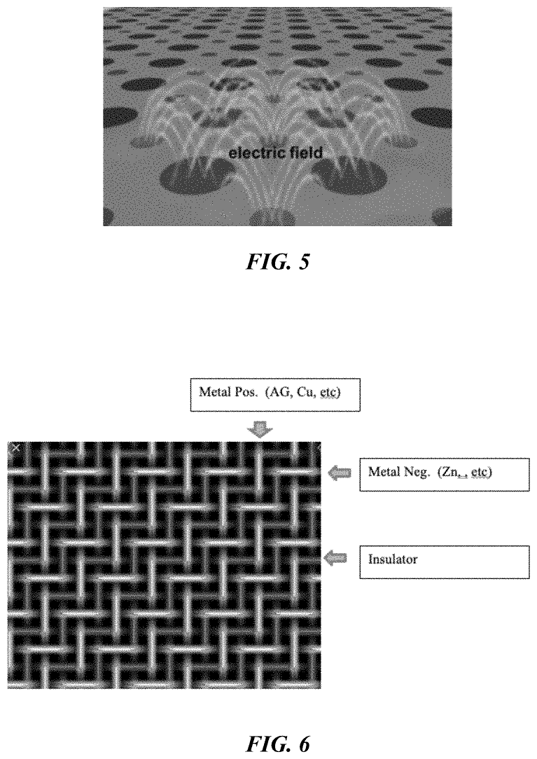

FIG. 5 shows a printed electric field array using printed, dissimilar metals to create a galvanic reaction resulting in an EMF between each cell pair wherein the distance between the galvanic cells can either increase or decrease the EMF within the user's wound.

FIG. 6 shows is example of a woven fiber print with different materials associated with the fibers wherein the fibers can be a negative and a positive wherein the positive can be silver, copper, and the negative can be zinc, aluminum, or the like. The two dissimilar metals can create a EMF within the user's skin and wound site.

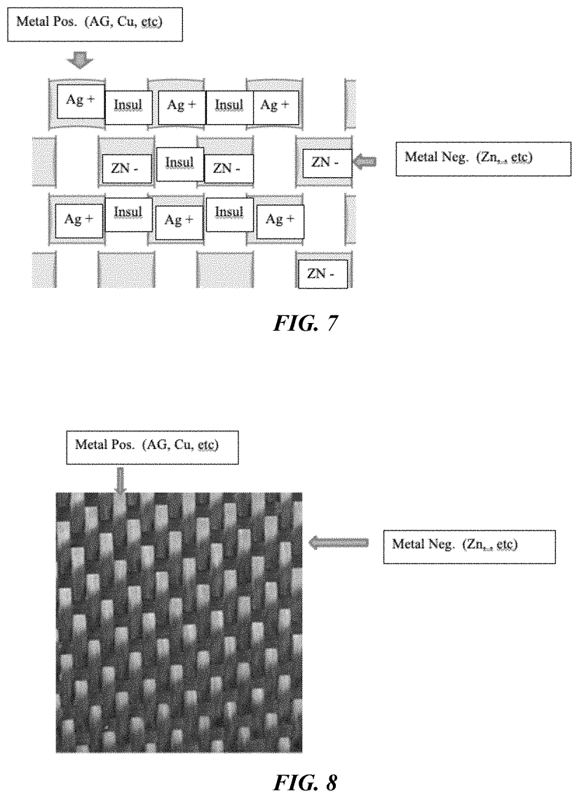

FIG. 7 is another example of a weave fiber print with different materials associated with the fibers

FIG. 8 is yet another example of an open weave pattern for keeping metal fibers physically separated by an insulated material wherein the non-active, insulated material can be non-conductive material such as cotton, ceramic, plastic, rubber, Teflon, or the like.

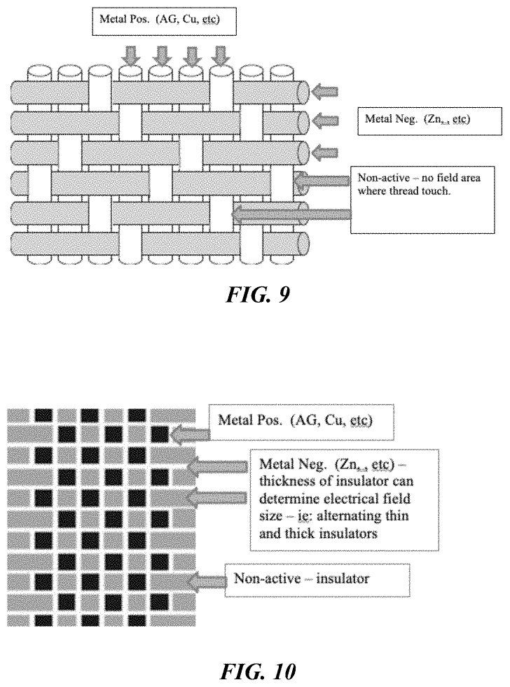

FIG. 9 is an example of a possible weave pattern for keeping metal fibers physically separated wherein the non-active material can vary in thickness creating varying EMF penetration in the user's skin or wound site.

FIG. 10 is another example of a possible weave pattern for keeping metal fibers physically separated by alternating insulators wherein the insulators can alternate by thick and thin insulators.

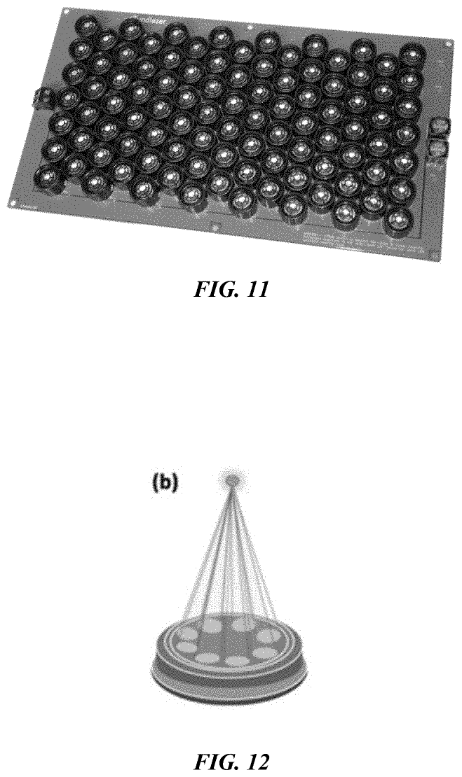

FIG. 11 shows a sonitrons ultrasound wherein the sonitrons ultrasound can be configured to transmit the ultrasonic field to the skin about a wound.

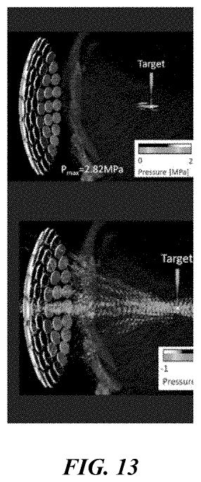

FIGS. 12-13 shows a focused ultrasound wherein the EMF cartridge in FIG. 1 can have a focused ultrasound that can focus the beams down into the user's skin and bite or sting site wherein the beams can be an array that shoot a voltage at an alternating current into the user skin's

In practice, when a person or animal is stung or struck with venom through the skin, the strike will have a puncture wound and will swell. The electrode is placed upon the site of puncture and swelling, and an electric field, ultrasound, or other form of energy is applied to the wound through the cartridge 14 as shown in FIG. 1. The application of the EMF or other energy denatures the proteins of the venom effectively nullifying the toxic effect of the venom in the person or animal. The cartridge 14 should be applied to the wound as soon as possible and may be applied until the person or animal experiences relief from the effects of the venom.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. A cartridge for applying an electromagnetic field (EMF) to at least one toxin in a
wound in the skin of a person or animal, the cartridge comprising:
a power supply;
a controller coupled to the power supply and providing a voltage between about 0.2V and 3V to an array of electrodes configured to transmit the EMF to the skin about the wound.

2. The cartridge according to claim 1 wherein the power supply is formed of dissimilar metals creating a galvanic reaction.

3. The cartridge according to claim 1 wherein the toxin comprises at least one protein and wherein the EMF denatures the protein.

4. A method of deactivating a toxin in a wound in the skin of a person or animal, the method comprising:
providing an electromagnetic field (EMF) to the wound with a voltage between about 0.2V and 3V through an array of electrodes configured to transmit the EMF to the skin about the wound.

5. The method of claim 4, wherein the toxin comprises at least one protein and wherein the EMF denatures the protein.

6. The method of claim 5, wherein the EMF renders the protein ineffective as a poison.

7. The cartridge according to claim 1, wherein the array of electrodes comprises a woven matrix of conductive threads spaced apart a distance to create the EMF.

8. The cartridge according to claim 1, wherein the power supply is a half cell reaction between a zinc thread and oxygen found within the skin.

9. A cartridge for applying an ultrasonic field to at least one toxin in a wound in the skin of a person or animal, the cartridge comprising:
a power supply;
a controller coupled to the power supply and providing a voltage between about 0.2V and 3V to an array of sonotrodes configured to transmit the ultrasonic field to the skin about the wound.

10. The cartridge of claim 1, further comprising a feedback circuit that measures impedance in the tissue and adjusts the electrical field based on the impedance.
